(19) United States
(12) Patent Application Publication
(10) Pub. No.: US 2017/0252094 A1
(43) Pub. Date: Sep. 7, 2017

(54) HEATING ENERGY TREATMENT SYSTEM AND CONTROL DEVICE

(71) Applicant: OLYMPUS CORPORATION, TOKYO (JP)

(72) Inventors: Yoshitaka HONDA, Hachioji-shi (JP); Norihiro YAMADA, Hino-shi (JP)

(73) Assignee: OLYMPUS CORPORATION, TOKYO (JP)

(21) Appl. No.: 15/261,139

(22) Filed: Sep. 9, 2016

Related U.S. Application Data
(63) Continuation of application No. PCT/JP2016/056816, filed on Mar. 4, 2016.

(51) Int. Cl. A61B 18/14; H05B 1/02

(52) U.S. Cl. CPC A61B 18/1445; H05B 1/025; A61B 2017/00734

(57) ABSTRACT

A control device for controlling a heat treatment device, wherein the heat treatment device includes: a heating resistor configured to be electrically connected to an energy output source to form a circuit such that the energy output source causes current flow through the heating resistor to generate heat for treating a target object, and wherein the control device includes: a detection circuit configured to detect one or more of a resistance component and a capacitance component of the circuit caused by a fluid; and one or more processors configured to control the energy output source to control a characteristic of the current flow through the heating resistor based on the one or more of the resistance component and the capacitance component detected by the detection circuit.

### CROSS-REFERENCE TO RELATED APPLICATION

This application is a Continuation Application of PCT International Application No. PCT/JP2016/056816, filed on Mar. 4, 2016. The entire content of PCT International Application No. PCT/JP2016/056816 is incorporated herein by reference.

### BACKGROUND

Technical Field

The present invention relates to a control device for controlling an energy output source to supply electric energy to a heating resistor provided in an energy treatment device, and a heating energy treatment system including the control device.

Background Art

International Publication No. WO 2011/089717 discloses a treatment system in which an energy control device supplies a radio frequency (RF) electric energy (alternating-current (AC) electric energy) to a heater provided in an energy treatment tool. In this treatment system, supply of RF electric energy to the heater causes the heater to generate heat. The heat generated by the heater is applied to a treatment target such as a biological tissue.

### SUMMARY

In a configuration in which a treatment is performed on a treatment target by using heat generated by a heater as disclosed in International Publication No. WO 2011/089717, a temperature of the heater is estimated based on a resistance value of the heater so that temperature control of the heater is performed in some cases. In such cases, the magnitude of AC electric energy to be supplied to the heater is controlled based on the resistance value of the heater so that the temperature of the heater is adjusted. In a treatment, fluid such as humor enters an area on an installation surface where the heater is installed (near the heater) to cause a short circuit of the heater or generate a capacitance component of fluid in some cases. In these cases, especially the capacitance component of fluid, for example, causes a phase difference between a current and a voltage output to the heater. As the phase difference between the current and the voltage increases, the influence on temperature control of the heater based on the resistance value of the heater increases.

The present invention has been made to solve problems described above, and has an object of providing an energy control device and an energy treatment tool that can perform appropriate temperature control on a heater based on a resistance value of the heater without an influence of entering of fluid into an area on an installation surface where the heater is installed (near the heater).

To achieve the object, an aspect of the present invention provides a heating energy treatment system comprising: a heat treatment device comprising: a heating resistor configured to be electrically connected to an energy output source to form a circuit such that the energy output source causes current flow through the heating resistor to generate heat for treating a target object; and a control device comprising: a detection circuit configured to detect one or more of a resistance component and a capacitance component of the circuit caused by a fluid; and one or more processors configured to control the energy output source to control a characteristic of the current flow through the heating resistor based on the one or more of the resistance component and the capacitance component detected by the detection circuit.

Another aspect of the present invention provides a control device for controlling a heat treatment device, wherein the heat treatment device comprises: a heating resistor configured to be electrically connected to an energy output source to form a circuit such that the energy output source causes current flow through the heating resistor to generate heat for treating a target object, and wherein the control device comprises: a detection circuit configured to detect one or more of a resistance component and a capacitance component of the circuit caused by a fluid; and one or more processors configured to control the energy output source to control a characteristic of the current flow through the heating resistor based on the one or more of the resistance component and the capacitance component detected by the detection circuit.

### BRIEF DESCRIPTION OF THE DRAWINGS

FIG. 1 schematically illustrates a treatment system according to a first embodiment.

FIG. 2 is a block diagram schematically illustrating a configuration in which the energy control device according to the first embodiment supplies energy to an energy treatment tool.

FIG. 3 schematically illustrates an example of a heater according to the first embodiment.

FIG. 4 is a flowchart illustrating a process of the energy control device in a treatment using heat generated by the heater in the first embodiment.

FIG. 5 schematically illustrates an example of a path of RF electric energy in a state where fluid has entered an area near the heater.

FIG. 6 schematically illustrates a state in which a phase difference occurs between a current and a voltage output to the heater.

FIG. 7 is a block diagram schematically illustrating a configuration in which an energy control device according to a first variation of the first embodiment supplies energy to an energy treatment tool.

FIG. 8 schematically illustrates an example of a matching circuit according to the first variation of the first embodiment in a state where a resistance component and a capacitance component due to fluid are generated in a heater.

FIG. 9 is a flowchart illustrating a process of an energy control device in a treatment using heat generated by a heater in a second variation of the first embodiment.

FIG. 10 schematically illustrates configurations of an installation surface where a heater is installed and an energy control device in a second embodiment.

FIG. 11 is a flowchart illustrating a process of the energy control device in a treatment using heat generated by the heater in the second embodiment.

FIG. 12 schematically illustrates a configuration of an installation surface where a heater is installed in a first variation of the second embodiment.

FIG. 13 schematically illustrates configurations of an installation surface where a heater is installed and an energy control device in a second variation of the second embodiment.

### DETAILED DESCRIPTION

### First Embodiment

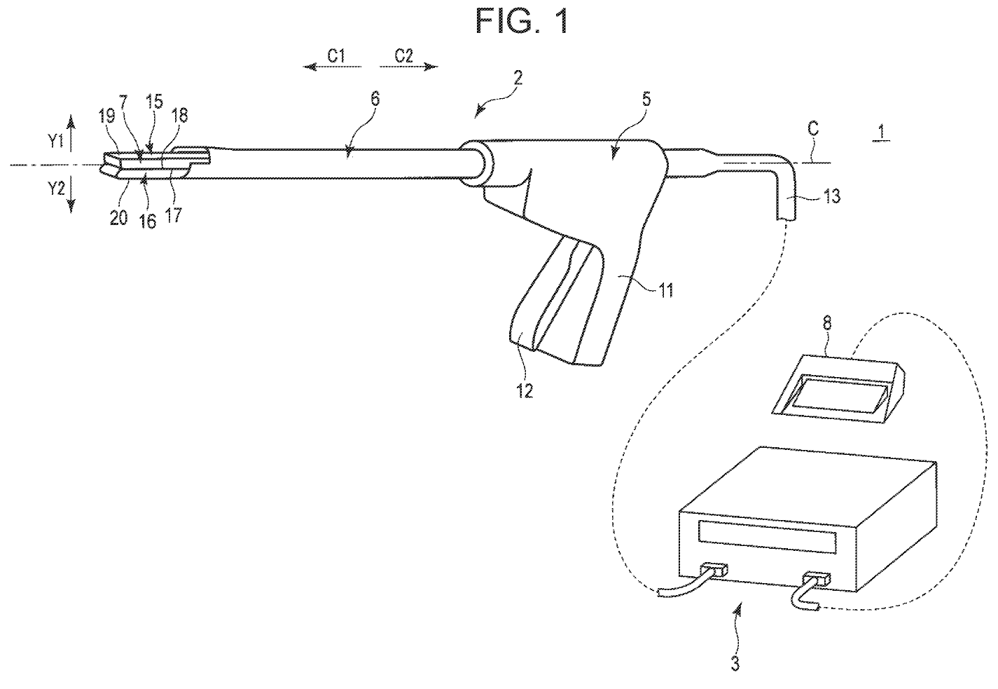

A first embodiment of the present invention will be described with reference to FIGS. 1 through 6.

FIG. 1 illustrates a treatment system 1 according to this embodiment. As illustrated in FIG. 1, the treatment system 1 includes an energy treatment tool 2 and an energy control device 3 that controls supply of energy to the energy treatment tool 2. The energy treatment tool 2 has a longitudinal axis C. Here, an end of a direction along the longitudinal axis C is defined as a distal end (indicated by arrow C1), and an end opposite to the distal end is defined as a proximal end (indicated by arrow C2).

The energy treatment tool 2 includes a housing 5 that can be grasped, a shaft 6 connected to the distal end of the housing 5, and an end effector 7 provided at the distal end of the shaft 6. The housing 5 includes a grip 11 to which a handle 12 is rotatably attached. When the handle 12 rotates relative to the housing 5, the handle 12 opens or closes relative to the grip 11.

The end effector 7 includes a first grasper 15 and a second grasper 16. When the handle 12 is opened or closed relative to the grip 11, a gap between the pair of the graspers 15 and 16 is opened or closed. In this manner, a treatment target such as a blood vessel (biological tissue) can be grasped between the pair of graspers 15 and 16. In FIG. 1, the opening/closing direction of the end effector 7 is indicated by arrows Y1 and Y2. The first grasper 15 has a first opposing surface (treatment surface) 17 opposed to the second grasper 16, and the second grasper 16 has a second opposing surface (treatment surface) 18 opposed to the first grasper 15 (first opposing surface 17). In a state where the treatment target is grasped between the graspers 15 and 16, the opposing surfaces 17 and 18 are in contact with the treatment target. An outer surface of the first grasper 15 includes a first rear face 19 facing in an opposite direction to the first opposing surface 17 in the opening/closing direction of the end effector 7. An outer surface of the second grasper 16 includes a second rear face 20 facing in an opposite direction to the second opposing surface 18 in the opening/closing direction of the end effector 7.

An end of a cable 13 is connected to the housing 5. The other end of the cable 13 is detachably connected to the energy control device 3. The treatment system 1 includes a foot switch 8 as an energy operation input part. The foot switch 8 receives an operation of causing the energy control device 3 to output energy to the energy treatment tool 2. Instead of or in addition to the foot switch 8, an operation button attached to the housing 5 of the energy treatment tool 2, for example, may be provided as an energy operation input part.

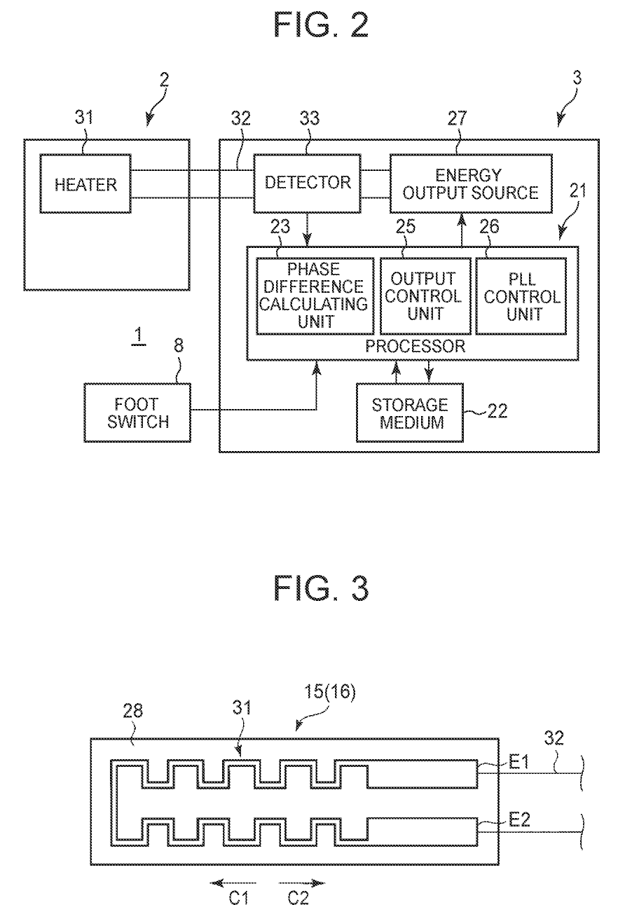

FIG. 2 illustrates a configuration in which the energy control device 3 supplies energy to the energy treatment tool 2. As illustrated in FIG. 2, the energy control device 3 includes a processor 21 that controls the entire treatment system and a storage medium 22. The processor (control unit) 21 is constituted by an integrated circuit including, for example, a central processing unit (CPU), an application specific integrated circuit (ASIC), or a field programmable gate array (FPGA). The processor 21 may be constituted by one integrated circuit or a plurality of integrated circuits. A processing in the processor 21 is performed in accordance with a program stored in the processor 21 or the storage medium 22. The storage medium 22 stores, for example, a processing program for use in the processor 21, a parameter and a table for use in computation in the processor 21. The processor 21 includes a phase difference calculating unit 23, an output control unit 25, and a phase lock loop (PLL) control unit 26. The phase difference calculating unit 23, the output control unit 25, and the PLL control unit 26 function as part of the processor 21, and perform part of a processing performed by the processor 21.

The energy control device 3 includes an energy output source 27 that outputs radio frequency (RF) electric energy that is AC electric power. The energy output source 27 includes, for example, a waveform generator, a conversion circuit, and a transformer (each not shown). The energy output source 27 converts electric power from a power supply (not shown) such as a battery or a plug socket to RF electric energy (AC electric energy), and outputs the obtained RF electric energy. The output control unit 25 of the processor 21 detects whether an operation input is performed with the energy operation input part such as the foot switch 8 or not. If the operation input is performed with, for example, the foot switch 8, the output control unit 25 causes the energy output source 27 to output RF electric energy. The output control unit 25 controls driving of the energy output source 27, and controls an output state of RF electric energy from the energy output source 27. In addition, the PLL control unit 26 adjusts a frequency f in the output of RF electric energy.

The energy treatment tool 2 includes a heater (heating element) 31. The heater 31 comprising a heating resistor configured to be electrically connected to an energy output source 27 to form a circuit such that the energy output source 27 causes current flow through the heating resistor to generate heat for treating a target object. In an embodiment, a heater 31 is provided in at least one of the graspers 15 and 16 in the end effector 7. FIG. 3 illustrates an example of the heater 31. As illustrated in FIG. 3, in an embodiment, the heater 31 is provided in at least one of the graspers 15 and 16, and in each of the graspers (15; 16; 15, 16) provided with the heater 31, the heater 31 is disposed on an installation surface 28. In each of the graspers (15; 16; 15, 16) provided with the heater 31, the installation surface 28 is disposed inside, and is sandwiched between the opposing surface (a corresponding one of the opposing surfaces 17 and 18) and the rear face (a corresponding one of the rear faces 19 and 20) in the opening/closing direction of the end effector 7. That is, in each of the graspers (15; 16; 15, 16) provided with the heater 31, the installation surface 28 is located at the side at which the end effector 7 opens relative to the opposing surface (a corresponding one of the opposing surfaces 17 and 18). The heater 31 includes connection ends E1 and E2, and extends to form a substantially U-shape, for example, between the connection ends E1 and E2.

As illustrated in FIG. 2, the heater 31 is electrically connected to the energy output source 27 through a supply path 32. RF electric energy (RF electric power) output from the energy output source 27 is supplied to the heater 31 through the supply path 32. While RF electric energy is being supplied to the heater 31, a potential difference occurs between the connection ends E1 and E2 in the heater 31 so that a current flows in the heater 31. In this manner, heat is generated in the heater 31. In each grasper provided with the heater 31, heat generated in the heater 31 is transmitted to the opposing surface (a corresponding one of the opposing surfaces 17 and 18) that is the treatment surface through the installation surface 28. In a state where the treatment target is grasped, heat is applied to the treatment target from the opposing surface (17; 18; 17, 18) to which the heat of the heater 31 is transmitted. The presence of the heater 31 in at least one of the graspers 15 and 16 enables heat generated by the heater 31 to be applied from at least one of the opposing surfaces 17 and 18 to the treatment target.

The supply path 32 from the energy output source 27 to the heater 31 is provided with a detector 33. The detector 33 is constituted by, for example, a current detecting circuit and a voltage detecting circuit provided in the energy control device 3, for example. In a state where RF electric energy is output from the energy output source 27, the detector 33 detects a current I and a voltage V output from the energy output source 27 to the heater 31. In this manner, chronological changes of the current I and the voltage V can be detected. Information on the current I and the voltage V detected by the detector 33 is converted from an analog signal to a digital signal by, for example, an A/D converter (not shown), and the resulting digital signal is transmitted to the processor 21.

The output control unit 25 calculates a resistance value R of the heater 31 based on detection results of the current I and the voltage V in the detector 33. In this manner, a chronological change of the resistance value R of the heater 31 can be detected. The resistance value R of the heater 31 changes in accordance with a temperature T of the heater 31. Thus, the output control unit 25 estimates the temperature T of the heater 31 based on the resistance value R of the heater 31 and a relationship between the resistance value R and the temperature T stored in, for example, the storage medium 22. Based on the estimated temperature T of the heater 31, the output control unit 25 controls an output state of RF electric energy from the energy output source 27, and performs temperature control of the heater 31. For example, in an embodiment, constant temperature control of chronologically keeping the temperature T of the heater 31 constant at a target temperature TO is performed by controlling the output state of RF electric energy from the energy output source 27 based on the resistance value R. In this manner, temperature control of the heater 31 based on the resistance value R of the heater 31 is performed in this embodiment.

Based on the detection result of the detector 33, the phase difference calculating unit 23 of the processor 21 calculates phase information of the current I output to the heater 31 and the voltage V output to the heater 31. Then, based on the phase information on the current I and the voltage V, the phase difference calculating unit 23 calculates a phase difference Δθ between the current I and the voltage V. In this manner, a chronological change of the phase difference Δθ can be detected. Based on the phase difference Δθ, the output control unit 25 of the processor 21 controls an output state of RF electric energy from the energy output source 27 and controls supply of RF electric energy to the heater 31. Based on the phase difference Δθ, the PLL control unit 26 adjusts frequency of the current I or the voltage V, and adjusts a frequency f in the output of RF electric energy (RF electric power).

Effects and advantages of the energy treatment tool 2 and the energy control device 3 according to this embodiment will now be described. In performing a treatment with the treatment system 1, the end effector 7 is inserted into a body cavity such as an abdominal cavity, and a treatment target (biological tissue) such as a blood vessel is placed between the graspers 15 and 16. Then, the handle 12 is closed relative to the grip 11 so that the gap between the graspers 15 and 16 is closed. In this manner, the treatment target is grasped between the graspers 15 and 16, and the opposing surfaces 17 and 18 contact the treatment target. In this state, an operation input is performed with the energy operation input part such as the foot switch 8 so that the energy output source 27 outputs RF electric energy (RF electric power). The output RF electric energy is supplied to the heater 31, and the heater 31 generates heat. In each of the graspers (15; 16; 15, 16) provided with the heater 31, heat generated by the heater 31 is applied to the treatment target grasp on the opposing surface (a corresponding one of the opposing surfaces 17 and 18). With application of heat generated by the heater 31 to the treatment target, the treatment target is solidified concurrently with dissection, and a treatment is performed on the treatment target using heat generated by the heater 31.

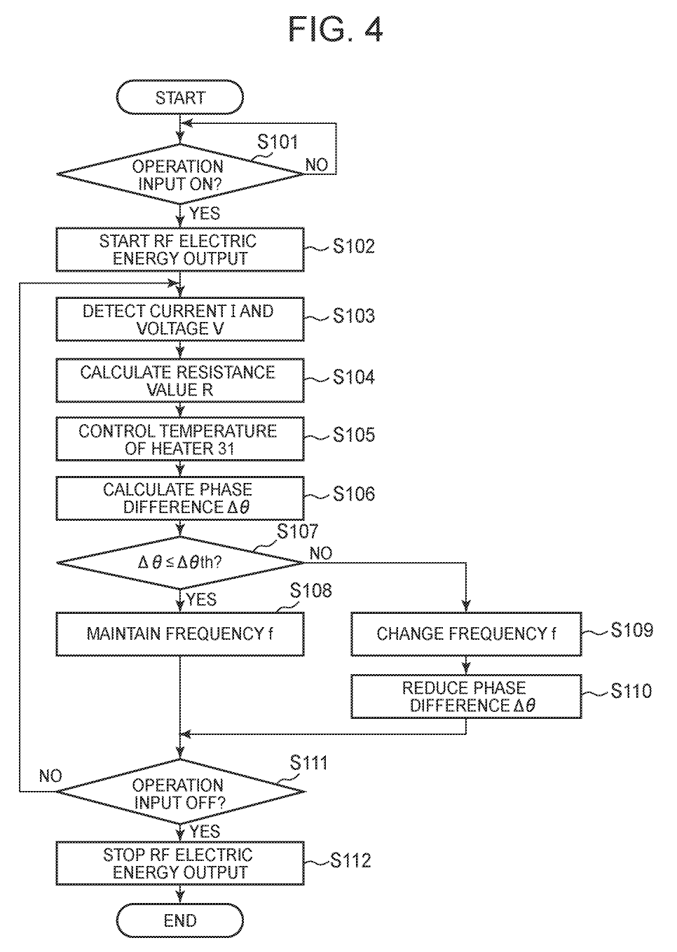

FIG. 4 is a flowchart illustrating a process of the energy control device 3 in a treatment using heat generated by the heater 31. As illustrated in FIG. 4, the processor 21 (output control unit 25) determines whether an operation input is performed with the foot switch (energy operation input part) 8 or not (i.e., whether an operation input is ON or OFF) (step S101). If the operation input is not performed (step S101—No), the process returns to step S101.

Specifically, the processor (control unit) 21 is kept on standby until an operation input is performed with the foot switch 8. If the operation input is performed (step S101—Yes), the processor 21 (output control unit 25) starts an output of RF electric energy from the energy output source 27 (step S102).

When the output of RF electric energy starts, the detector 33 detects a current I and a voltage V output from the energy output source 27 to the heater 31 (step S103). Based on detection results of the current I and the voltage V, the processor 21 (output control unit 25) calculates a resistance value R of the heater 31 (step S104). Based on the calculated resistance value R, the processor 21 (output control unit 25) controls an output state of RF electric energy from the energy output source 27 and performs temperature control of the heater 31 (step S105).

When an output of RF electric energy starts, the processor 21 (phase difference calculating unit 23) calculates phase information on the current I and the voltage V, and calculates a phase difference Δθ between the current I and the voltage V (step S106). Then, the processor 21 (phase difference calculating unit 23) determines whether the calculated phase difference Δθ is less than or equal to a predetermined threshold Δθth (whether Δθ≦Δθth) or not (step S107). If the phase difference Δθ is less than or equal to the predetermined threshold Δθth (step S107—Yes), the processor 21 (PLL control unit 26) maintains a frequency f in the output of RF electric energy (step S108).

On the other hand, if the phase difference Δθ is larger than the predetermined threshold Δθth (step S107—No), the processor 21 (PLL control unit 26) changes the frequency f in the output of RF electric energy with PLL control (step S109) to reduce the phase difference Δθ (step S110). That is, the processor 21 performs control of reducing the phase difference Δθ by adjusting the frequency f. For example, if the phase difference Δθ is larger than the predetermined threshold Δθth, the frequency f in the output of RF electric energy is reduced so that the phase difference Δθ is reduced.

Once the process of step S108 or the process of step S110 has been performed, the processor 21 (output control unit 25) determines whether the operation input with the foot switch 8 is kept ON or not (step S111). While the operation input is kept ON (step S111—No), the process returns to step S103, and the process of step S103 and subsequent processes are sequentially performed. If the operation input is switched to OFF (step S111—Yes), the processor 21 (output control unit 25) stops the output of RF electric energy from the energy output source 27 (step S112). In this embodiment, through the processes performed in the foregoing manner, the control of maintaining the phase difference Δθ at the predetermined threshold Δθth or less is performed while RF electric energy is output.

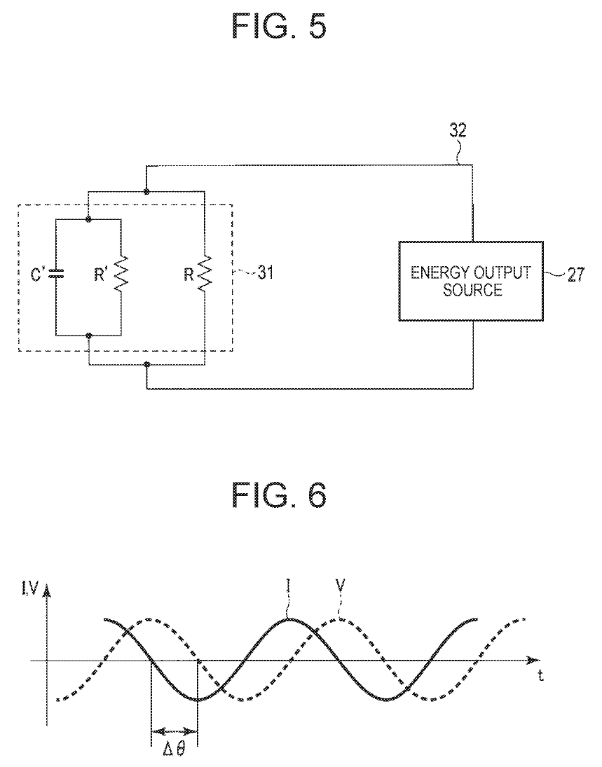

In a treatment, fluid such as humor might enter an inside of the graspers 15 and 16. The fluid might enter an area on an installation surface 28 where the heater 31 is installed (near the heater 31) so that the state on the installation surface 28 changes. The fluid that has entered an area on the installation surface 28 can cause a short circuit in the heater 31 or generate a capacitance component of fluid. FIG. 5 illustrates an example of a path of RF electric energy output from the energy output source 27 in a state where fluid has entered an area (on the installation surface 28) near the heater 31. As illustrated in FIG. 5, when fluid enters the area near the heater 31, a resistance component R′ of fluid and a capacitance component C′ of fluid, for example, are generated in a circuit (supply path 32) of RF electric energy output from the energy output source 27. In this case, especially the capacitance component C′ of fluid causes a phase difference Δθ between a current I and a voltage V output to the heater 31. FIG. 6 illustrates a state where a phase difference occurs between the current I and the voltage V. In FIG. 6, the abscissa represents time t and ordinate represents the current I and the voltage V. In FIG. 6, a chronological change of the current I is indicated by a solid line, and a chronological change of the voltage V is indicated by a broken line. As the phase difference Δθ increases, the influence on calculation of the resistance value R of the heater 31 based on the current I and the voltage V increases, and the influence on temperature control based on the resistance value R increases.

In this embodiment, as described above, in the process of step S106, the phase difference Δθ is calculated, and in the process of step S107, it is determined whether the phase difference Δθ is less than or equal to the predetermined threshold Δθth or not (whether Δθ≦Δθth or not). If the phase difference Δθ is larger than the predetermined threshold Δθth, the processes of step S109 and S110 are performed with PLL control. That is, a process (control) of changing the frequency f in the output of RF electric energy to reduce the phase difference Δθ. The process of reducing the phase difference Δθ by changing the frequency f is repeatedly performed chronologically until the phase difference Δθ is reduced to the predetermined threshold Δθth or less. The predetermined threshold Δθth is such a small value that the phase difference Δθ hardly affects calculation of the resistance value R of the heater 31 based on the current I and the voltage V, for example. In an embodiment, the predetermined threshold Δθth may be set at 0. In the case where the predetermined threshold Δθth is 0, the processes of step S109 and S110 are performed until the current I and the voltage V come to be in the same phase.

As described above, in this embodiment, when the phase difference Δθ between the current I and the voltage V increases, the frequency f in the output of RF electric energy is changed so that the phase difference Δθ is reduced. Accordingly, the influence of the phase difference Δθ decreases, and the processor 21 can appropriately calculates the resistance value R of the heater 31 based on the current I and the voltage V. In this manner, the processor 21 appropriately controls an output state of RF electric energy from the energy output source 27 based on the resistance value R of the heater 31 so that temperature control of the heater 31 based on the resistance value R can be accurately performed with stability. Thus, the temperature control of the heater 31 based on the resistance value R of the heater 31 can be appropriately performed without influence of entering of fluid into an area on the installation surface 28 where the heater 31 is installed (a change of the state on the installation surface 28).

### Variations of First Embodiment

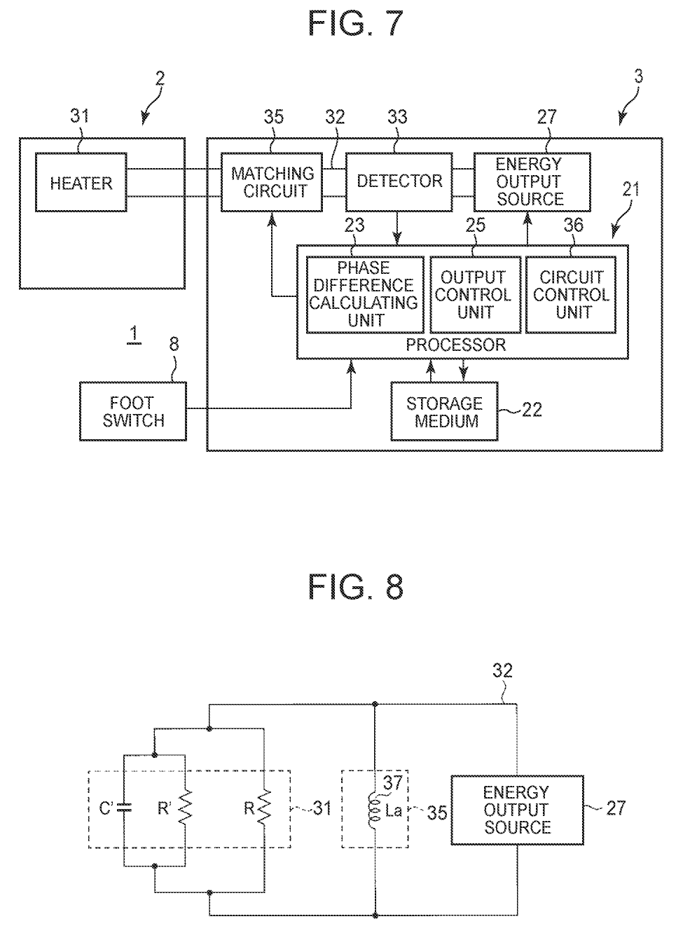

In the first embodiment, the phase difference Δθth is reduced by changing the frequency f. The present invention, however, is not limited to this example. For example, as illustrated in FIGS. 7 and 8 as a first variation of the first embodiment, a matching circuit 35 may be provided in the supply path 32 of RF electric energy from the energy output source 27 to the heater 31. FIG. 7 illustrates a configuration in which the energy control device 3 supplies energy to the energy treatment tool 2 in this variation. As illustrated in FIG. 7, in this variation, the processor 21 includes a circuit control unit 36 that controls driving of the matching circuit 35. The circuit control unit 36 constitutes part of the processor 21, and performs part of the process performed by the processor 21. The circuit control unit 36 controls driving of the matching circuit 35 based on the phase difference Δθ. In this variation, PLL control described in the first embodiment is not performed.

FIG. 8 illustrates an example of the matching circuit 35 in a state where a resistance component R′ and a capacitance component C′ due to fluid are generated in the heater 31. In the embodiment illustrated in FIG. 8, a variable coil 37 is disposed electrically in parallel to the heater 31 (heater resistance) in the matching circuit 35. The variable coil 37 has a variable inductance La. In the embodiment illustrated in FIG. 8, the circuit control unit 36 adjusts an inductance La of the variable coil 37 in the matching circuit 35 based on the phase difference Δθ.

In this variation, in a manner similar to the first embodiment, in a state where the energy output source 27 outputs RF electric energy to the heater 31, the detector 33 detects a current I and a voltage V output to the heater 31 (step S103 in FIG. 4). In this variation, in a manner similar to the first embodiment, the processor 21 calculates a resistance value R of the heater 31 (step S104 in FIG. 4), and performs temperature control of the heater 31 based on the resistance value R (step S105 in FIG. 4). In this variation, in a manner similar to the first embodiment, the processor 21 calculates a phase difference Δθ (step S106 in FIG. 4), and determines whether the phase difference Δθ is less than or equal to a predetermined threshold Δθth or not (step S107 in FIG. 4).

Note that in this variation, if the phase difference Δθ is less than or equal to the predetermined threshold Δθth (step S107—Yes), the processor 21 (circuit control unit 36) maintains the inductance La of the variable coil 37 in step S108. On the other hand, if the phase difference Δθ is larger than the predetermined threshold Δθth (step S107—No), the processor 21 (circuit control unit 36) controls driving of the matching circuit 35 to change the inductance La of the variable coil 37 in step S109. In this manner, the processor 21 reduces the phase difference Δθ (step S110 in FIG. 4). That is, the processor 21 performs control of reducing the phase difference Δθ by adjusting the inductance La of the variable coil 37. For example, if the phase difference Δθ is larger than the predetermined threshold Δθth, the processor 21 reduces the inductance La of the variable coil 37 to reduce the phase difference Δθ. In this variation, through the process as described above, control of maintaining the phase difference Δθ at the predetermined threshold Δθth or less is also performed in a state where RF electric energy is output.

As described above, in this variation, when the phase difference Δθ between the current I and the voltage V increases, the inductance La of the variable coil 37 is changed so that the phase difference Δθ is reduced. By performing control of reducing the phase difference Δθ, advantages similar to those in the first embodiment can be obtained in this variation.

In the variation illustrated in FIGS. 7 and 8, the variable coil 37 is provided electrically in parallel to the heater 31 in the matching circuit 35. The present invention, however, is not limited to this example. For example, in a variation, instead of or in addition to the variable coil 37, the matching circuit 35 may include a variable capacitor having a variable capacitance. In this variation, when the phase difference Δθ exceeds the predetermined threshold Δθth (step S107—No), the processor 21 (circuit control unit 36) controls driving of the matching circuit 35 to change the capacitance of the variable capacitor in step S109. In this manner, the processor 21 reduces the phase difference Δθ (step S110). In another variation, in the matching circuit 35, a variable coil and/or a variable capacitor may be electrically connected to the heater 31 in series. In this case, the processor 21 also adjusts the capacitance of an inductance of the variable coil and/or a capacitance of the variable capacitor based on the phase difference Δθ.

In another variation, the processor 21 may perform both adjustment of a frequency f in an output of RF electric energy and control of driving of the matching circuit 35 based on the phase difference Δθ. In this variation, when the phase difference Δθ exceeds the predetermined threshold Δθth (step S107—No), the processor 21 changes the frequency f in the output of RF electric energy and changes the inductance La of variable coil 37 and/or the capacitance of the variable capacitor in the matching circuit 35 in step S109. In this manner, the processor 21 reduces the phase difference Δθ (step S110).

In the first embodiment and embodiments described above including the first variation, if the phase difference Δθ between the current I and the voltage V is larger than the predetermined threshold Δθth, the processor 21 performs control of reducing the phase difference Δθ. That is, the processor 21 performs control of maintaining the phase difference Δθ at the predetermined threshold Δθth or less.

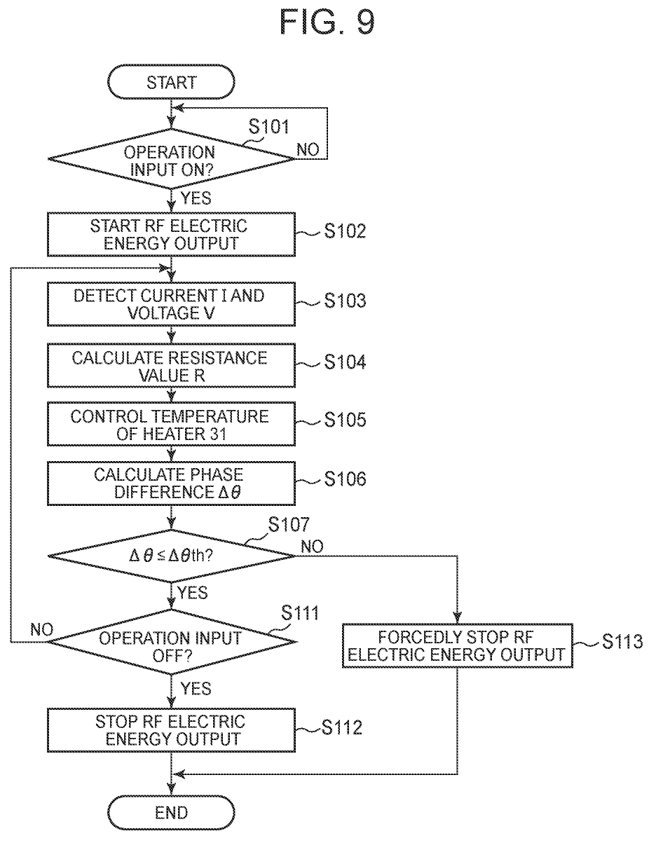

In a second variation of the first embodiment illustrated in FIG. 9, control of reducing the phase difference Δθ is not performed. FIG. 9 is a flowchart illustrating a process of the energy control device 3 in a treatment using heat generated by the heater 31 in this variation. As illustrated in FIG. 9, in this variation, processes of steps S101 to S107 are performed in a manner similar to the first embodiment. Note that in this variation, after determination in step S107, only in a case where the phase difference Δθ is the predetermined threshold Δθth or less (step S107—Yes), the processor 21 determines whether the operation input is kept ON with the foot switch 8 or not (step S111). As long as the operation input is kept ON (step S111—No), the process returns to step S103, and the process of step S103 and subsequent processes are performed again. If the operation input is switched to OFF (step S111—Yes), the processor 21 (output control unit 25) stops the output of RF electric energy from the energy output source 27 (step S112).

On the other hand, if the phase difference Δθ is larger than the predetermined threshold Δθth (step S107—No), the processor 21 forcedly stops an output of RF electric energy from the energy output source 27 (step S113). That is, based on a situation where the phase difference Δθ is larger than the predetermined threshold Δθth, the processor 21 stops an output of RF electric energy from the energy output source 27.

As described above, in this variation, when the phase difference Δθ between the current I and the voltage V increases, the output of RF electric energy is stopped. In this manner, when fluid enters an area on the installation surface 28 (near the heater 31), the output of RF electric energy is stopped. Thus, in a manner similar to the first embodiment, temperature control of the heater 31 based on the resistance value R of the heater 31 can be appropriately performed without influence of entering of fluid into an area on the installation surface 28 where the heater 31 is installed.

In the first embodiment and variations thereof, the energy control device 3 includes: the energy output source 27 that outputs RF electric energy (AC electric energy) to be supplied to the heater 31; and the detector 33 that detects a current I and a voltage V output from the energy output source 27 to the heater 31 in a state where the energy output source 27 outputs RF electric energy (AC electric energy). The energy control device 3 also includes the processor 21 that calculates a phase difference Δθ between the current I and the voltage V output to the heater 31 based on a detection result of the detector 33 and controls supply of RF electric energy to the heater 31 based on the phase difference Δθ.

### Second Embodiment

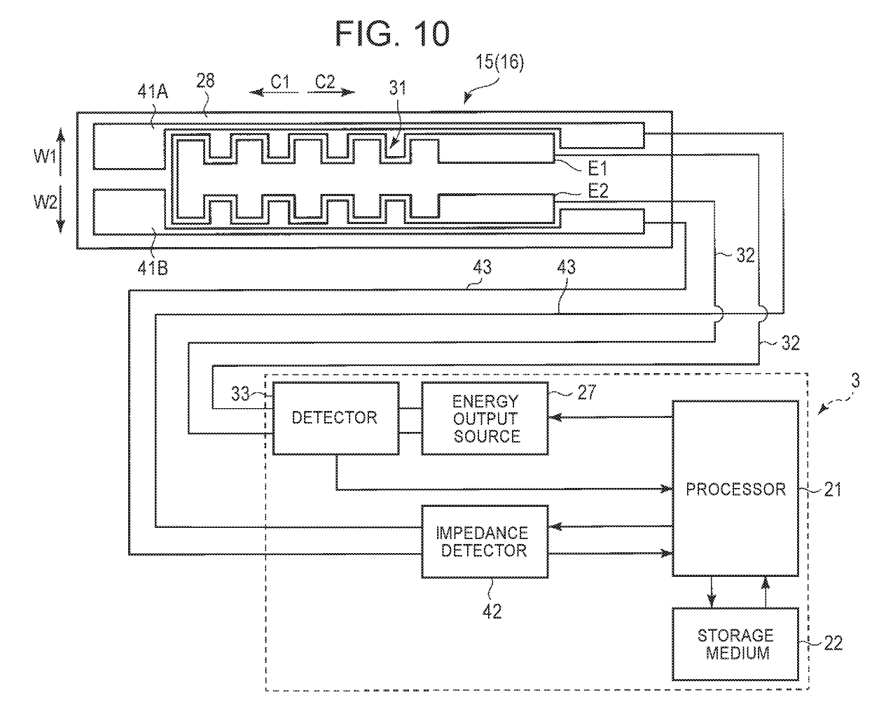

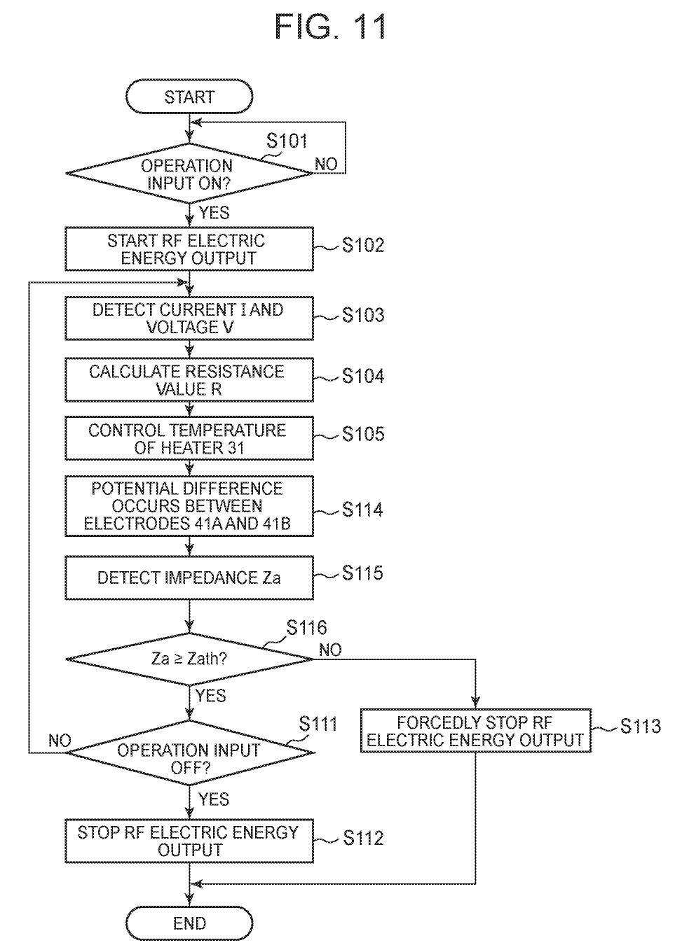

A second embodiment of the present invention will now be described with reference to FIGS. 10 and 11. The second embodiment is obtained by modifying the configuration of the first embodiment as described below. The same reference numerals designate the same components in the first embodiment, and description thereof will not be repeated.

FIG. 10 schematically illustrates configurations of the installation surface 28 where the heater 31 is installed (near the heater 31) and the energy control device 3 in this embodiment. As illustrated in FIG. 10, in this embodiment, a pair of electrodes 41A and 41B are provided on the installation surface 28 where the heater 31 is installed. Here, intersecting directions (directions indicated by arrows W1 and W2) intersecting with a longitudinal axis C are defined. The intersecting directions (substantially perpendicularly) intersect with, for example, the longitudinal axis C, and (substantially perpendicularly) intersect with opening/closing directions of an end effector 7 (directions indicated by arrows Y1 and Y2 in FIG. 1). The electrode 41A encloses the heater 31 at a distal end (indicated by arrow C1) and one end (indicated by arrow W1) in the intersecting direction on the installation surface 28. The electrode 41B encloses the heater 31 at the distal end (indicated by arrow C1) and the other end (indicated by arrow W2) in the intersecting direction on the installation surface 28. On the installation surface 28, each of the electrodes 41A and 41B is located outside the heater 31.

In this embodiment, in a manner similar to the first embodiment, the energy control device 3 includes a processor 21, a storage medium 22, and an energy output source 27. The energy output source 27 is electrically connected to the heater 31 through a supply path 32. In this embodiment, the energy output source 27 also supplies RF electric energy (AC electric energy) to the heater 31 so that the heater 31 generates heat. Using the heat generated by the heater 31, a treatment is performed on a treatment target. A detector 33 that detects a current I and a voltage V output from the energy output source 27 to the heater 31 is also provided.

In this embodiment, the processor 21 calculates a resistance value R of the heater 31 based on detection results of the current I and the voltage V in the detector 33. Based on the calculated resistance value R, the processor 21 (output control unit 25) estimates a temperature T of the heater 31 and performs temperature control of the heater 31. Note that in this embodiment, unlike the first embodiment, a phase difference Δθ between the current I and the voltage V is not calculated.

In this embodiment, the energy control device 3 includes an impedance detector (detector) 42 that detects an impedance Za between the electrodes 41A and 41B. The impedance detector 42 is electrically connected to the electrodes 41A and 41B through a measurement path 43. The impedance detector 42 includes, for example, a conversion circuit, a transformer, and an integrated circuit (each not shown), and the integrated circuit includes, for example, a detection circuit and an arithmetic circuit. Here, the integrated circuit provided in the impedance detector 42 may function as part of the processor 21.

The impedance detector 42 converts electric power from a power supply (not shown) to electric energy for measurement (measurement electric power) that is electric energy different from RF electric energy, and outputs the obtained measurement electric energy. The output measurement electric energy is supplied to the electrodes 41A and 41B through the measurement path 43. The supply of the measurement electric energy to the electrodes 41A and 41B causes a potential difference between the electrodes 41A and 41B. The power supply that supplies electric power to the impedance detector 42 may be the same as the power supply of the energy output source 27 and may be different from the power supply of the energy output source 27. An output of measurement electric energy from the impedance detector 42 is controlled by the processor 21.

The impedance detector 42 measures a current flowing in the measurement path 43 and a potential difference between the pair of electrodes 41A and 41B, for example. Based on the measurement results, the impedance detector 42 detects (calculates) an impedance Za between the electrodes 41A and 41B. In this manner, a chronological change of the impedance Za is detected, and the impedance Za is monitored. In this embodiment, in a state where the energy output source 27 outputs RF electric energy, the output state of RF electric energy from the energy output source 27 is controlled based on the detection result of the impedance Za in the impedance detector 42, and supply of RF electric energy to the heater 31 is controlled.

FIG. 11 is a flowchart illustrating a process of the energy control device 3 in a treatment using heat generated by the heater 31 in this embodiment. As illustrated in FIG. 11, in this embodiment, processes of steps S101 to S105 are performed in a manner similar to the first embodiment. In this embodiment, however, calculation of a phase difference Δθ between the current I and the voltage V (step S106) is not performed, and determination based on the phase difference Δθ (step S107) is not performed, either.

In this embodiment, in a state where the energy output source 27 outputs RF electric energy to the heater 31, the processor 21 causes the impedance detector 42 to output measurement electric energy to the pair of electrodes 41A and 41B so that a potential difference is generated between the electrodes 41A and 41B (step S114). Then, the impedance detector (detector) 42 detects an impedance Za between the electrodes 41A and 41B based on, for example, the potential difference between the electrodes 41A and 41B and a current flowing in the measurement path 43 (step S115).

The processor 21 determines whether the impedance Za detected by the impedance detector 42 is greater than or equal to a predetermined threshold Zath (whether Za Zath) or not (step S116). If the impedance Za is greater than or equal to the predetermined threshold Zath (step S116—Yes), the processor 21 determines whether an operation input is kept ON with a foot switch 8 or not (step S111). As long as the operation input is kept ON (step S111—No), the process returns to step S103, and the process of step S103 and subsequent processes are performed again. If the operation input is switched to OFF (step S111—Yes), the processor 21 (output control unit 25) stops the output of RF electric energy from the energy output source 27 (step S112).

On the other hand, if the impedance Za is smaller than the predetermined threshold Zath (step S116—No), the processor 21 forcedly stops the output of RF electric energy from the energy output source 27 (step S113). That is, based on a situation where the impedance Za is smaller than the predetermined threshold Zath, the processor 21 stops the output of RF electric energy from the energy output source 27.

In this embodiment, in a manner similar to the first embodiment, fluid such as humor can enter an area on the installation surface 28 where the heater 31 is installed (near the heater 31) in some cases. Fluid that has entered the area on the installation surface 28 can cause a short circuit in the heater 31 or generate a capacitance component of fluid. In this case, in a manner similar to the first embodiment, a phase difference Δθ occurs between a current I and a voltage V output to the heater 31.

In this embodiment, when fluid enters an area on the installation surface 28, the electrodes 41A and 41B become electrically conductive through the fluid. The electrical conduction between the electrodes 41A and 41B reduces the impedance Za between the electrodes 41A and 41B. That is, the impedance Za between the electrodes 41A and 41B changes in accordance with a change of the state of entering of fluid into an area on the installation surface 28 (i.e., a change of the state on the installation surface 28).

In this state, the impedance Za is calculated through the process of step S115, and it is determined whether the impedance Za is greater than or equal to the predetermined threshold Zath or not through the process of step S116, as described above. If the impedance Za is smaller than the predetermined threshold Zath, the output of RF electric energy from the energy output source 27 is forcedly stopped through the process of step S113.

Since control is performed as described above, in this embodiment, when fluid enters an area on the installation surface 28 so that a short circuit or a capacitance component of fluid, for example, is generated in the heater 31, the output of RF electric energy is appropriately stopped. Thus, in this embodiment, in a manner similar to the first embodiment, temperature control of the heater 31 based on the resistance value R of the heater 31 can be appropriately performed without influence of entering of fluid into an area on the installation surface 28 where the heater 31 is installed.

### Variations of Second Embodiment

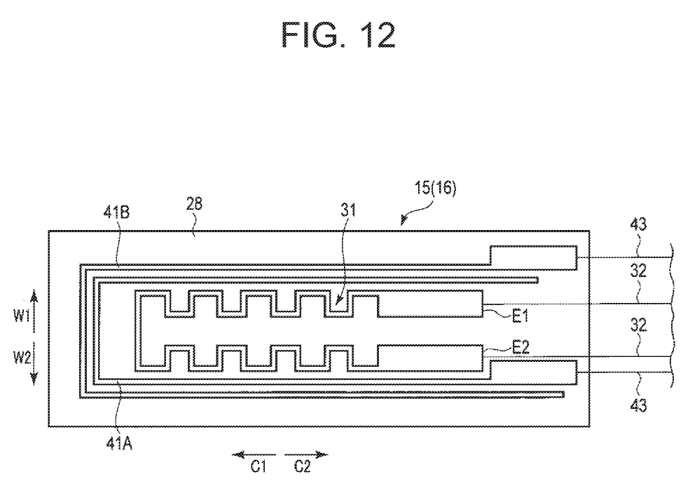

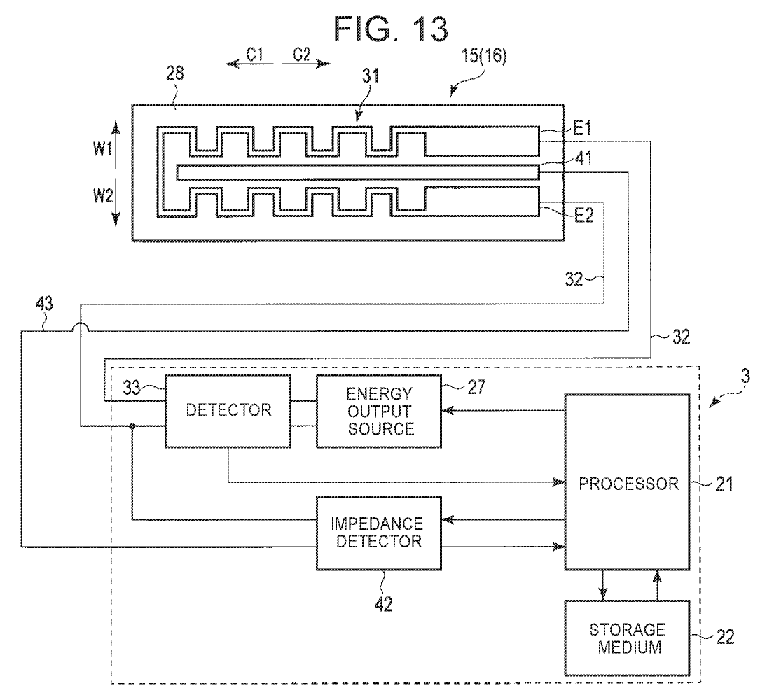

Arrangement of the electrodes 41A and 41B on the installation surface 28 is not limited to the arrangement described in the second embodiment. For example, in a first variation of the second embodiment illustrated in FIG. 12, on the installation surface 28, the electrode 41A encloses the heater 31 at the distal end (indicated by arrow C1) and at both sides in the intersecting directions (indicated by arrows W1 and W2) intersecting with the longitudinal axis C. The other electrode 41B encloses the electrode 41A at the distal end and at both sides in the intersecting direction. Thus, on the installation surface 28, the electrode 41A is located outside the heater 31, and the electrode 41B is located outside the heater 31 and the electrode 41A. In this variation, in a treatment using heat generated by the heater 31, the energy control device 3 performs processes similar to those in the second embodiment (see FIG. 11).

In this variation, with the arrangement of the electrodes 41A and 41B as described above, when fluid enters an area on the installation surface 28, the electrodes 41A and 41B become electrically conductive through the fluid before a short circuit caused by the fluid or a capacitance component of fluid, for example, is generated in the heater 31. Thus, before a short circuit or a capacitance component of fluid, for example, is generated in the heater 31, it is determined that the impedance Za is smaller than the predetermined threshold Zath in step S116, and an output of RF electric energy from the energy output source 27 is stopped through the process of step S113. That is, in this variation, when fluid enters an area on the installation surface 28 (near the heater 31), the entering of fluid is promptly and accurately detected so that detection accuracy can be enhanced.

In the second embodiment and the first variation thereof, the pair of electrodes 41A and 41B are disposed on the installation surface 28. The present invention, however, is not limited to this example. For example, in a second variation of the second embodiment illustrated in FIG. 13, only one electrode 41 is provided on the installation surface 28. In this variation, on the installation surface 28, the heater 31 encloses the electrode 41 at the distal end (indicated by arrow C1) and both sides in the intersecting directions (indicated by arrows W1 and W2) intersecting with the longitudinal axis C. Thus, on the installation surface 28, the heater 31 is disposed outside the electrode 41.

In this variation, the impedance detector 42 is electrically connected to the heater 31 and the electrode 41 through the measurement path 43. Thus, in this variation, part of the supply path 32 is shared as the measurement path 43. In this variation, measurement electric energy is not output from the impedance detector 42, but energy output source 27 outputs RF electric energy to the heater 31 so that a potential difference occurs between the heater 31 and the electrode 41. At this time, in an embodiment, the electrode 41 has substantially the same potential as one connection end E1 of the heater 31, and a potential difference between the other connection end E2 and the electrode 41 is at the maximum in the heater 31.

In this variation, the impedance detector 42 measures a current flowing in the measurement path 43 and a potential difference between the electrode 41 and the heater 31, for example. Based on the measurement results, the impedance detector 42 detects (calculates) an impedance Zb between the electrode 41 and the heater 31. In this manner, a chronological change of the impedance Zb is detected, and the impedance Zb is monitored. In this variation, in a state where the energy output source 27 outputs RF electric energy, the output state of RF electric energy from the energy output source 27 is controlled based on the detection result of the impedance Zb in the impedance detector 42, and supply of RF electric energy to the heater 31 is controlled.

In this variation, processes of steps S101 to S105 are performed in a manner similar to the second embodiment (see FIG. 11). Note that in this variation, in step S114, the processor 21 generates a potential difference between the electrode 41 and the heater 31. Then, the impedance detector (detector) 42 detects the impedance Zb between the electrode 41 and the heater 31 in step S115.

In addition, in this variation, in step S116, the processor 21 detects whether the impedance Zb detected by the impedance detector 42 is greater than or equal to a predetermined threshold Zbth (whether Zb≧Zbth) or not. If the impedance Zb is greater than or equal to the predetermined threshold Zbth (step S116—Yes), the process proceeds to step S111. On the other hand, if the impedance Zb is smaller than the predetermined threshold Zbth (step S116—No), the process proceeds to step S113, and the processor 21 forcedly stops the output of RF electric energy from the energy output source 27. That is, based on a situation where the impedance Zb is smaller than the predetermined threshold Zbth, the processor 21 stops the output of RF electric energy from the energy output source 27.

In this variation, with the arrangement of the electrode 41 and the heater 31 as described above, when fluid enters an area on the installation surface 28, the electrode 41 and the heater 31 become electrically conductive through the fluid before a short circuit caused by fluid or a capacitance component of fluid, for example, is generated in the heater 31. Thus, before a short circuit or a capacitance component of fluid, for example, is generated in the heater 31, it is determined that the impedance Zb is smaller than the predetermined threshold Zbth in step S116, and the output of RF electric energy from the energy output source 27 is stopped through the process of step S113. That is, in this variation, when fluid enters an area on the installation surface 28 (near the heater 31), the entering of fluid is promptly and accurately detected so that detection accuracy can be enhanced.

In the variation illustrated in FIG. 11, the electrode 41 is disposed inside the heater 31. Alternatively, in another variation, the electrode 41 may be disposed outside the heater 31.

In the second embodiment and the variations thereof, if the impedance Za is smaller than the predetermined threshold Za or if the impedance Zb is smaller than the predetermined threshold Zb, the output of RF electric energy is stopped. The present invention, however, is not limited to this example. For example, in a variation, if the impedance Za is smaller than the predetermined threshold Za or if the impedance Zb is smaller than the predetermined threshold Zb, a frequency f in the output of RF electric energy may be changed by the PLL control described above. In another variation, a matching circuit 35 may be provided in the supply path 32. In this variation, if the impedance Za is smaller than the predetermined threshold Za or if the impedance Zb is smaller than the predetermined threshold Zb, the matching circuit 35 changes an inductance La of a variable coil 37 and/or a capacitance of a variable capacitor.

In the second embodiment and the variations thereof, the energy control device 3 includes: the energy output source 27 that outputs RF electric energy (AC electric energy) to be supplied to the heater 31; and the detector 42 that detects an impedance Za between the pair of electrodes 41A and 41B provided on the installation surface 28 where the heater 31 is installed or an impedance Zb between the electrode 41 and the heater 31 provided on the installation surface 28. The energy control device 3 also includes the processor 21 that controls supply of RF electric energy to the heater 31 based on the impedance Za between the electrodes 41A and 41B and the impedance Zb between the electrode 41 and the heater 31 detected by the detector 42.

### Other Variations

In the foregoing embodiments, for example, RF electric energy is supplied to the heater 31. The present invention, however, is not limited to this example. For example, in a case where low frequency electric energy is supplied to the heater 31 as AC electric energy, control described above is also applicable.

In the embodiments described above, for example, only heat generated by the heater 31 is applied to the treatment target. The present invention, however, is not limited to this example. For example, in a variation, RF current is applied to the treatment target that is grasped as well as heat generated by the heater 31. In this variation, a treatment electrode (not shown) is provided in each of the graspers 15 and 16, and RF electric energy different from RF electric energy supplied to the heater 31 is supplied to the treatment electrodes. In a treatment, each of the treatment electrodes contacts the treatment target that is grasped. Thus, RF electric energy is supplied to each of the treatment electrodes while the treatment target is grasped so that RF current flows between the treatment electrodes through the treatment target and is applied to the treatment target.

In another variation, ultrasonic vibrations are applied to the treatment target that is grasped as well as heat generated by the heater 31. In this variation, an ultrasonic transducer (not shown) is provided in the energy treatment tool 2 so that electric energy (e.g., AC power an output of which has a predetermined frequency) different from RF electric energy supplied to the heater 31 is supplied to the ultrasonic transducer. In this manner, ultrasonic vibrations are generated in the ultrasonic transducer and transmitted to one of the graspers 15 and 16. The ultrasonic vibrations are transmitted to one of the graspers 15 and 16 with the treatment target being grasped so that the transmitted ultrasonic vibrations are applied to the treatment target.

In the embodiments described above, for example, the end effector 7 includes the pair of graspers 15 and 16. The present invention, however, is not limited to this example. In a variation, the end effector 7 is formed in a hook shape, a spatula shape, or a blade shape, for example. In this case, in a treatment, the end effector 7 is brought into contact with the treatment target, and heat generated by the heater 31 is applied to the treatment target. At this time, ultrasonic vibrations may be transmitted to the end effector 7 so that ultrasonic vibrations are applied to the treatment target as well as heat generated by the heater 31. RF current may be caused to flow through the treatment target between a treatment electrode provided in the end effector 7 and a neutral electrode placed outside the body. In this case, RF current is applied to the treatment target as well as heat generated by the heater 31.

The embodiments of the present invention, for example, have been described. The present invention, however, is not limited to these embodiments, and various changes or modifications may be, of course, made within the scope of the invention.

What is claimed is:

1. A heating energy treatment system comprising:
a heat treatment device comprising:
a heating resistor configured to be electrically connected to an energy output source to form a circuit such that the energy output source causes current flow through the heating resistor to generate heat for treating a target object; and
a control device comprising:
a detection circuit configured to detect one or more of a resistance component and a capacitance component of the circuit caused by a fluid; and
one or more processors configured to control the energy output source to control a characteristic of the current flow through the heating resistor based on the one or more of the resistance component and the capacitance component detected by the detection circuit.

2. The heating energy treatment system according to claim 1,
wherein the detection circuit is configured to detect one or more of the resistance component and the capacitance component caused by the fluid by detecting the current flow and a voltage output by the energy output source to the heating resister in a state where the energy output source outputs AC electric energy.

3. The heating energy treatment system according to claim 2,
wherein the one or more processors are configured to:
calculate a phase difference between the current flow and the voltage output from the energy output source detected by the detection circuit; and
control supply of the AC electric energy to the heating resister based on the phase difference.

4. The heating energy treatment system according to claim 3,
wherein the one or more processors are configured to:
determine whether the phase difference is larger than a predetermined threshold; and
perform control of reducing the phase difference in response to determining that the phase difference is larger than the predetermined threshold.

5. The heating energy treatment system according to claim 4,
wherein the one or more processors are configured to perform the control of reducing the phase difference by changing a frequency of the AC electric energy output by the energy output source.

6. The heating energy treatment system according to claim 4, further comprising:
a matching circuit comprising at least one of a variable coil or a variable capacitor, wherein the matching circuit is arranged in the circuit on a supply path of the AC electric energy from the energy output source to the heating resistor,
wherein the one or more processors are configured to perform the control of reducing the phase difference by changing an inductance of the variable coil and/or a capacitance of the variable capacitor in the matching circuit.

7. The heating energy treatment system according to claim 3,
wherein the one or more processors are configured to:
determine whether the phase difference is larger than a predetermined threshold; and
control the energy output source to stop output of the AC electric energy in response to determining that the phase difference is larger than the predetermined threshold.

8. The heating energy treatment system according to claim 1,
wherein the detection circuit is configured to detect one or more of the resistance component and the capacitance component caused by the fluid by detecting either an impedance between a pair of electrodes provided on an installation surface where the heating resistor is installed or an impedance between an electrode on the installation surface and the heating resistor.

9. The heating energy treatment system according to claim 8,
wherein the one or more processors are configured to control the energy output source to supply AC electric energy to the heating resistor based on the impedance between the pair of electrodes or the impedance between the electrode and the heating resistor detected by the detection circuit.

10. The heating energy treatment system according to claim 9,
wherein the one or more processors are configured to:
determine whether the impedance between the pair of electrodes or the impedance between the electrode and the heating resistor is smaller than a predetermined threshold; and
control the energy output source to stop output of the AC electric energy in response to detecting that the impedance between the pair of electrodes or the impedance between the electrode and the heating resistor is smaller than the predetermined threshold.

11. The heating energy treatment system according to claim 1,
wherein the heat treatment device further comprises:
an installation surface, wherein the heating resistor is installed on the installation surface; and
a treatment surface configured to receive the heat, wherein the treatment surface is configured to supply the heat to the target object, and
wherein the heat treatment device further comprises either:
a pair of electrodes provided on the installation surface, wherein an impedance between the pair of electrodes is configured to vary in accordance with a change of the one or more of the resistance component and the capacitance component, or
an electrode provided on the installation surface, wherein an impedance between the electrode and the heating resistor is configured to vary in accordance with the change of the one or more of the resistance component and the capacitance component.

12. The energy treatment system according to claim 11,
wherein each of the pair of electrodes is located outside of the heating resistor on the installation surface.

13. A control device for controlling a heat treatment device,
wherein the heat treatment device comprises:
a heating resistor configured to be electrically connected to an energy output source to form a circuit such that the energy output source causes current flow through the heating resistor to generate heat for treating a target object, and
wherein the control device comprises:
a detection circuit configured to detect one or more of a resistance component and a capacitance component of the circuit caused by a fluid; and
one or more processors configured to control the energy output source to control a characteristic of the current flow through the heating resistor based on the one or more of the resistance component and the capacitance component detected by the detection circuit.

14. A control device for controlling a heat treatment device,
wherein the heat treatment device comprises:
a heating resistor configured to be electrically connected to an energy output source to form a circuit such that the energy output source causes current flow through the heating resistor to generate heat for treating a target object, and
wherein the control device comprises:
a detection means for detecting one or more of a resistance component and a capacitance component of the circuit caused by a fluid; and
a processing means for controlling the energy output source to control a characteristic of the current flow through the heating resistor based on the one or more of the resistance component and the capacitance component detected by the detection means.
